(19) United States
(12) Patent Application Publication
(10) Pub. No.: US 2019/0311812 A1
(43) Pub. Date: Oct. 10, 2019

(54) ADVANCED HEALTH MONITORING SYSTEM AND METHOD

(71) Applicant: MOBILE INNOVATIONS LLC, Parkville, MO (US)

(72) Inventor: Robert J. Sweeney, Kansas City, MO (US)

(21) Appl. No.: 16/162,575

(22) Filed: Oct. 17, 2018

Related U.S. Application Data
(60) Provisional application No. 62/655,447, filed on Apr. 10, 2018.

(51) Int. Cl. G16H 80/00; G16H 10/60; G16H 15/00; A61B 5/00; A61B 5/024; G06T 11/20

(52) U.S. Cl. CPC G16H 80/00; G16H 10/60; G06T 11/206; A61B 5/0022; A61B 5/024; G16H 15/00

(57) ABSTRACT

A health monitoring (HM) computing device for monitoring patient vitals in real time is provided. The HM computing device includes at least one processor in communication with at least one memory device. The at least one processor is programmed to receive patient data from a plurality of patient computer devices associated with a plurality of patients. The at least one processor is also programmed to, for each patient of the plurality of patients, compare the received patient data corresponding to the patient to a reference model tailored to the corresponding patient. The at least one processor is also programmed to determine, based upon the comparisons, at least one status category for each of the plurality of patients. Moreover, the at least one processor is further programmed to generate and transmit instructions for displaying a first graphical user interface to a healthcare provider computer device associated with the healthcare provider.

### CROSS-REFERENCE TO RELATED APPLICATIONS

This application claims the benefit of priority to U.S. Provisional Patent Application No. 62/655,447, filed Apr. 10, 2018, entitled “ADVANCED HEALTH MONITORING SYSTEM AND METHOD,” the entire contents and disclosure of which are hereby incorporated by reference herein in their entirety.

### BACKGROUND

The field of the invention relates generally to a health monitoring system and, more particularly, to a network based system and method for tracking patients and managing patient-provided data in real time.

Many health conditions and diseases, such as heart disease, require active care and continuous attention by not only the patient, but also by the patient's healthcare provider, such as a doctor and/or nurse. Patients may often struggle to reach their healthcare provider when faced with a medical problem that requires immediate attention. Some patients may be readmitted to a healthcare facility (e.g., hospital) and incur additional expenses for medical concerns that could have been addressed remotely by their healthcare provider. At least some known systems attempt to facilitate interactive communication between a remote patient and their healthcare provider after a patient is discharged by requiring a patient to communicate with their healthcare provider on a daily basis. However, some of these known systems are overly complicated, expensive, and difficult to use by both patients and healthcare providers who are managing multiple patients on daily basis. Accordingly, there exists a need for a real-time patient monitoring system that enables patients to effectively communicate with healthcare providers on a daily basis, and enables healthcare providers to accurately manage patient data for multiple patients in real time.

### BRIEF DESCRIPTION

In one aspect, a health monitoring (HM) computing device for monitoring patient vitals in real time is provided. The HM computing device includes at least one processor in communication with at least one memory device. The at least one processor is programmed to receive patient data from a plurality of patient computer devices associated with a plurality of patients. The patient data includes measurements for at least one of weight, blood pressure, and pulse. The at least one processor is also programmed to, for each patient of the plurality of patients, compare the received patient data corresponding to the patient to a reference model tailored to the corresponding patient. The reference model is based on historical patient data of the corresponding patient, and includes threshold data for the at least one of weight, blood pressure, and pulse, the threshold data established by a healthcare provider associated with the corresponding patient. The at least one processor is further programmed to determine, based upon the comparisons, at least one status category for each of the plurality of patients. The at least one processor is also programmed to generate instructions for displaying a first graphical user interface on a healthcare provider computer device associated with the healthcare provider. Moreover, the at least one processor is programmed to transmit the generated instructions to a healthcare provider computer device associated with the healthcare provider to cause the first graphical user interface to be displayed on the healthcare provider computer device.

In another aspect, a computer-based method for monitoring patient vitals in real time is provided. The method is implemented using a health monitoring (HM) computing device. The HM computing device includes at least one processor in communication with at least one memory device. The method includes receiving, by the HM computing device, patient data from a plurality of user computer devices associated with a plurality of patients. The patient data includes measurements for at least one of weight, blood pressure, and pulse. The method also includes, for each patient of the plurality of patients, comparing, by the HM computing device, the received patient data corresponding to the patient to a reference model tailored to the corresponding patient. The reference model is based on historical patient data of the corresponding patient, and including threshold data for the at least one of weight, blood pressure, and pulse. The threshold data is established by a healthcare provider associated with the corresponding patient. The method further includes determining, based upon the comparisons, at least one status category for each of the plurality of patients. The method also includes generating instructions for displaying a first graphical user interface on a healthcare provider computer device associated with the healthcare provider. The method further includes transmitting the generated instructions to the healthcare provider computer device to cause the first graphical user interface to be displayed on the healthcare provider computer device.

In yet another aspect, one or more non-transitory computer-readable storage media having computer-executable instructions embodied thereon is provided. When executed by at least one processor on a health monitoring (HM) computing device, the computer-executable instructions cause the at least one processor to receive patient data from a plurality of user computer devices associated with a plurality of patients. The patient data includes measurements for at least one of weight, blood pressure, and pulse. When executed by the at least one processor, the computer-executable instructions further cause the at least one processor to, for each patient of the plurality of patients, compare the received patient data corresponding to the patient to a reference model tailored to the corresponding patient. The reference model is based on historical patient data of the corresponding patient, and includes threshold data for the at least one of weight, blood pressure, and pulse. The threshold data is established by a healthcare provider associated with the corresponding patient. When executed by the at least one processor, the computer-executable instructions also cause the at least one processor to determine, based upon the comparisons, at least one status category for each of the plurality of patients. The computer-executable instructions also cause the at least one processor to generate instructions for displaying a first graphical user interface on a healthcare provider computer device associated with the healthcare provider. The computer-executable instructions further cause the at least one processor to transmit the generated instructions to the healthcare provider computer device to cause the first graphical user interface to be displayed on the healthcare provider computer device.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and are instrumentalities shown, wherein:

FIG. 1 is an example screenshot of a patient home screen from the HM computing device for a patient user interface in accordance with an example embodiment of the present disclosure.

FIG. 2A is an example screenshot of a daily measurement screen from the HM computing device for a patient user interface in accordance with an example embodiment of the present disclosure.

FIG. 2B is an example screenshot of a patient question screen from the HM computing device for a patient user interface in accordance with an example embodiment of the present disclosure.

FIG. 3 is an example screenshot of a patient report screen from the HM computing device for a patient user interface in accordance with an example embodiment of the present disclosure.

FIG. 4 is an example screenshot of a patient communication screen from the HM computing device for a patient user interface in accordance with an example embodiment of the present disclosure.

FIG. 5 is an example screenshot of a patient reports from the HM computing device for a healthcare provider user interface in accordance with an example embodiment of the present disclosure.

FIG. 6 is an example screenshot of a first visual representation screen from the HM computing device for a healthcare provider user interface in accordance with an example embodiment of the present disclosure.

FIG. 7A is an example screenshot of a second visual representation screen from the HM computing device for a healthcare provider user interface in accordance with an example embodiment of the present disclosure.

FIG. 7B is another example screenshot of a second visual representation screen from the HM computing device for a healthcare provider user interface in accordance with an example embodiment of the present disclosure.

FIG. 8 is an example screenshot of a third visual representation screen from the HM computing device for a healthcare provider user interface in accordance with an example embodiment of the present disclosure.

FIG. 9 is an example screenshot of a fourth visual representation screen from the HM computing device for a healthcare provider user interface in accordance with an example embodiment of the present disclosure.

FIG. 10 is a flowchart of an example process for providing a health monitoring (HM) computer system using the system shown in FIG. 11, in accordance with an example embodiment of the present disclosure.

FIG. 11 is a simplified block diagram of an example health monitoring (HM) computer system for implementing the process shown in FIG. 10, in accordance with one embodiment of the present disclosure.

FIG. 12 is an example configuration of a client computer device as shown in FIG. 11, in accordance with an example embodiment of the present disclosure.

FIG. 13 is an example configuration of a server system as shown in FIG. 11, in accordance with an example embodiment of the present disclosure.

### DETAILED DESCRIPTION OF THE DISCLOSURE

The systems and methods described herein are directed to a health monitoring (HM) system that includes a health monitoring (HM) computing device for monitoring patient vitals in real time. In the example embodiment, a HM system includes a patient (e.g., patient or a caretaker acting on behalf of patient) who provides daily vital measurements (e.g., key marker measurements), and the patient's healthcare provider (e.g., attending physician, nurse, and/or healthcare professional) who monitors the patient's condition in real time based on the provided daily vital measurements.

In the example embodiment, the HM computing device generates a daily measurement screen for a patient user interface. The daily measurement screen enables a patient to track and report their (e.g., key markers) to their healthcare provider every day. The daily measurement screen enables the patient to input daily measurements for weight, blood pressure, and pulse in corresponding input fields. The HM computing device determines whether the inputted measurements are within a baseline status category, a warning status category, or a critical status category. In the example embodiment, the baseline status category refers to ideal and/or normal measurements for a patient. The warning status category refers to inputted measurements that exceed the baseline status category, and are trending towards the critical status category. The critical status category refers to inputted measurements that exceed the warning status category, and require immediate action to be taken by the patient and/or the patient's healthcare provider. Each status category corresponds to thresholds that are tailored to each patient's medical history, medications, and needs. The thresholds are set by the patient's healthcare provider, and are adjustable based on the patient's medical needs and circumstances.

When a patient inputs measurements into an input field, a corresponding status indicator changes from a first color to a second color to alert the patient, in real time, as to whether the inputted measurement is categorized as baseline, warning, or critical. For example, the status indicator next to each input field may be a grey color as default. In this example, when the patient inputs blood pressure measurements into a corresponding field, the corresponding status indicator may change from the grey color to a green color, indicating that the inputted blood pressure measurements are within the baseline status category. In further embodiments, the HM computing device subsequently prompts the patient to provide details as to the patient's condition. The HM computing device may display, each day, questions as to the patient's mood, symptoms, and/or medication.

In the example embodiment, the HM computing device generates a visual representation screen for a healthcare provider user interface. The visual representation screen may display an interactive graphical representation, such as an interactive pie chart. The interactive pie chart may include patient data for all patients in a hospital facility and/or a hospital department (e.g., specialty, unit). Each slice of the interactive pie chart corresponds to one of the three status categories. In the example embodiment, a healthcare provider may select a slice, for example, a slice corresponding to the critical status category, to view all the patients who need immediate attention. The healthcare provider may view a total number of patients within the critical status category, and the patient data associated with each patient. Based on information provided by the visual representation screen, healthcare providers are able to assess the status of all their patients on one screen, and allocate time and resources efficiently, by reaching out to patients who require immediate medical attention before addressing the needs of their other patients.

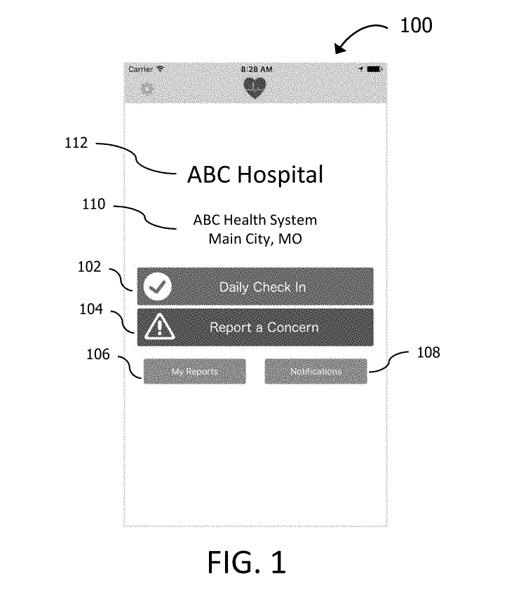

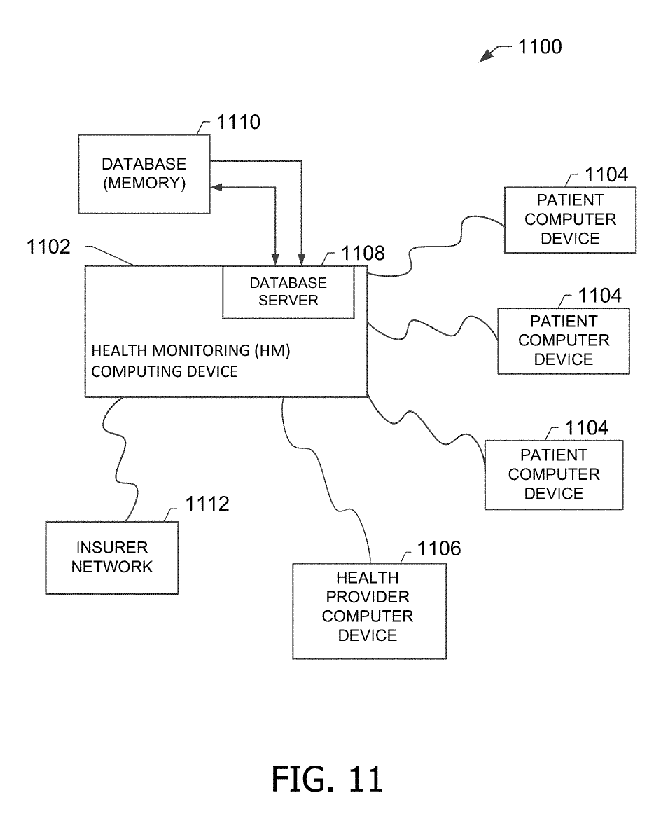

FIGS. 1-4 illustrate example screenshots of a patient user interface in accordance with example embodiments of the present disclosure. More specifically, FIG. 1 is an example screenshot of a patient home screen 100 from an HM computing device 1102 (shown in FIG. 11) for a patient user interface in accordance with an example embodiment of the present disclosure. In the example embodiment, a patient is registered with HM computing device 1102 by their healthcare provider (e.g., nurse, physician, and/or health professional). Patient home screen 100 is displayed on a patient computer device, such as patient computer device 1104 (shown in FIG. 11).

As shown in FIG. 1, HM computing device 1102 displays a “daily check in” button 102 that enables a patient to access HM computing device 1102, and report their daily key markers (e.g., weight, blood pressure, and pulse). In some embodiments, when the patient selects “daily check in” button 102, HM computing device 1102 displays a login page (not shown), prompting the patient to input their username and password. In other embodiments, the patient is directed to a login page prior to accessing patient home screen 100. In certain embodiments, the patient is only prompted to input their username and password during registration, for example, when the patient is downloading a software application (“app”) associated with the service(s) provided by HM computing device 1102 onto their patient computer device. In these embodiments, the patient's username and password are encrypted.

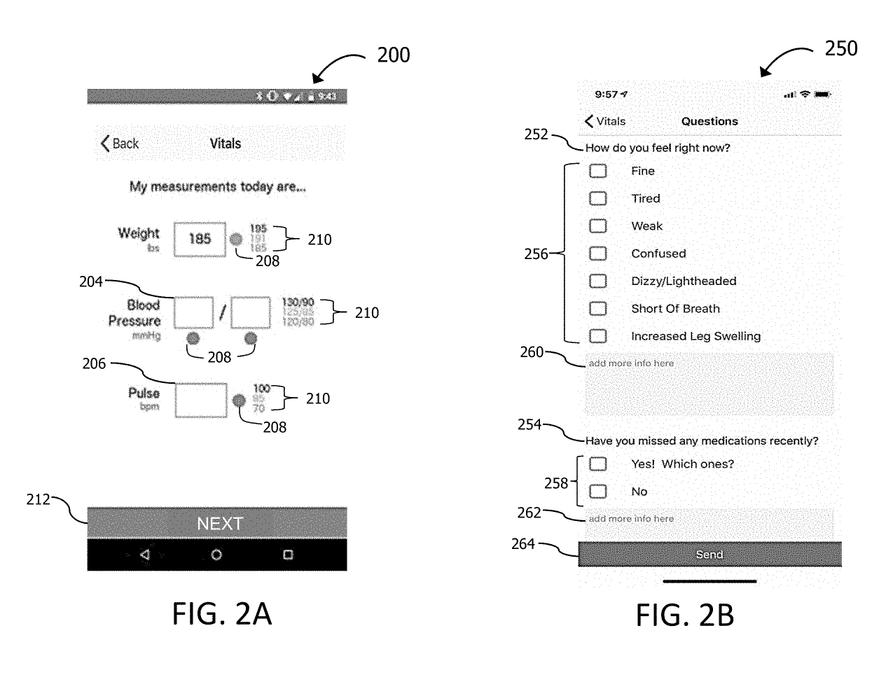

The patient may select button 102 to report, for a given date and time, how they are feeling (e.g., fine, tired, weak, dizzy/lightheaded, confused), their symptoms (e.g., shortness of breath, increased leg swelling), inquiries as to additional health concerns (e.g., diabetes, pulmonary cancer), and any additional information the patient would like their healthcare provider to know. In the example embodiment, as described below with regard to FIG. 2B, HM computing device 1102 displays general questions relevant to the patient's disease state and condition (e.g., symptoms, mood) each time the patient selects button 102 to check in and report their daily key markers. In other embodiments, HM computing device 1102 may display questions in response to concerns submitted by the patient. The patient user interface also provides a “report a concern” button 104 on patient home screen 100, which allows the patient to report a concern and/or submit a question relevant to their disease state. For example, the patient can select “report a concern” button 104 to send a message to their healthcare provider in regards to their symptoms and/or medication. In other embodiments, the patient may also select button 104 to submit additional concerns and/or questions that are not related to their diagnosed disease state. For example, if the patient selects button 104 to report an additional health concern, HM computing device 1102 may display questions that are relevant to the reported concern, such as diabetes.

The patient user interface further provides a “my reports” button 106 that enables the patient to view a log of their past reports. A patient's past reports include historical data as to the patient's activity with HM computing device 1102, including inputted measurements, reported concerns, and/or communications with the patient's healthcare provider. In the example embodiment, the patient user interface also provides a “notifications” button 108 that enables the patient to manage notifications (e.g., push notifications, messages, and/or daily reminders) sent by their healthcare provider. For example, by selecting “notifications” button 108, the patient may be directed to a notifications page (not shown) and/or a settings page that allows the patient to customize their push notifications.

In some embodiments, the patient receives an email containing a customized registration link when the patient's healthcare provider initially registers the patient with HM computing device 1102. The customized registration link enables the patient to automatically download the app associated with HM computing device 1102. The customized registration link may include a unique patient identifier (e.g., patient identification number, patient name) that associates the downloaded app with the patient. In other embodiments, the customized registration link may enable the patient to download and automatically log into the app without the patient having to manually input their username and password. The customized registration link may include an encrypted username and password associated with the patient. In these embodiments, the patient does not need to separately input their username and password each time they access the app. In embodiments where the patient logs out of the app, the patient may request a new secure login link via email. In these embodiments, after the patient logs out, HM computing device 1102 may display a login request page (not shown) on patient computer device 1104, prompting the patient to input the email address registered with HM computing device 1102. HM computing device 1102 may subsequently transmit a new login link to the patient's registered email address, thereby enabling the patient to automatically log back into the app.

In the example embodiment, HM computing device 1102 provides healthcare provider information to the patient on patient home screen 100. Patient home screen 100 includes a clickable healthcare provider display 110 (e.g., name, logo, and/or brand). Healthcare provider display 110 is that of a hospital and/or a health system associated with the patient. For example, healthcare provider display 110 may be that of a hospital at which the user is receiving medical attention for conditions such as congestive heart failure (“CHF”). By selecting healthcare provider display 110, HM computing device 1102 directs the patient to a website associated with the patient's healthcare provider. For example, the patient may be directed to the hospital's main website, a specific hospital department's website, and/or the hospital's patient portal website. Patient home screen 100 may further display the healthcare provider's address 112 and/or contact number (not shown).

In further embodiments, an emergency “911” button (not shown) may be provided on patient home screen 100. In these embodiments, the emergency “911” button enables the patient to call “911” by clicking a button provided on patient home screen 100. In other embodiments, an on-demand call button (not shown) may be provided on patient home screen 100. In these embodiments, the on-demand call button is a click-to-call button that enables the patient to directly contact their healthcare provider by selecting the button. In some embodiments, patient home screen 100 may include a scheduler button (not shown) that enables the patient to schedule an appointment with their healthcare provider. In further embodiments, patient home screen 100 may include a calendar button (not shown) that enables the patient to view time-sensitive information such as the patient's doctor's appointments, medication reminders (e.g., reminding the patient to take their medication and/or refill their prescription), and/or patient events/classes (e.g., meetings and/or classes relating to specific medical condition(s) associated with the patient).

FIG. 2A is an example screenshot of a daily measurement screen 200 from HM computing device 1102 for a patient user interface in accordance with an example embodiment of the present disclosure. Upon selecting “daily check in” button 102, HM computing device 1102 displays daily measurement screen 200, which enables the patient to input their daily vitals (e.g., key markers). More specifically, daily measurement screen 200 allows the patient to input information regarding their weight in pounds (“lbs”), blood pressure in millimeters of mercury (“mmHg”), and pulse in beats per minute (“bpm”) via weight input field 202, blood pressure input field 204, and pulse input field 206. When the patient inputs their key marker measurements in input fields 202, 204, and/or 206, a corresponding status indicator 208 changes from a first color to a second color.

In the example embodiment, a range 210 of thresholds is provided for each of the three key markers (e.g., weight, blood pressure, and pulse) next to each corresponding input field 202, 204, and 206. For each key marker, thresholds are set for the following three categories: baseline (e.g., ideal and/or normal measurements based on the patient's vitals), warning (e.g., measurements that exceed the baseline category, but not enough to cause alarm), and critical (e.g., measurements that can be life-threatening to the patient if immediate action is not taken). Thresholds are set by the patient's healthcare provider based on factors such as the patient's charts, medical history, and/or the most recent measurements taken by the patient's healthcare provider. Thus, HM computing device 1102 stores reference models tailored to each patient registered with HM computing device 1102 in a database such as database 1110 (shown in FIG. 11). Each patient's reference model includes range 210 of thresholds for each key marker set by the patient's healthcare provider based on the patient's medical history.

In the example embodiment, a patient's thresholds for weight can be based on the patient's most recent weigh-in measurements taken by the healthcare provider. For example, a healthcare provider may establish the patient's baseline weight as the most recent weigh-in measurement at the doctor's office, the patient's warning weight as weight gain in excess of the weigh-in measurement, and the patient's critical weight as significant weight gain in excess of the weigh-in measurement. The distinction between warning weight and critical weight is set by the patient's healthcare provider. For example, based on the patient's medical history, the patient's healthcare provider may consider a weight gain of 10 pounds as cause for concern, but a weight gain of 5 pounds to merely raise caution. The thresholds can be adjusted by the patient's healthcare provider based on factors such as weight loss, lifestyle changes (e.g., starting an exercise regime, dietary changes/restrictions, reducing alcohol and/or tobacco use), medication use, and/or surgery. The thresholds for one or more key marker may be updated by the patient's healthcare provider from their healthcare provider computer device, such as healthcare provider computer device 1140 (shown in FIG. 11) subsequent to, for example, a doctor's appointment.

In the example embodiment, each status category is associated with a color to provide a real-time visual representation to the patient as the patient inputs their daily measurement for each key marker. For example, baseline measurements may be green, warning measurements may be yellow, and critical measurements may be red. In the example embodiment, as the patient inputs a measurement into input fields 202, 204, and/or 206, a corresponding status indicator 208 automatically changes from a default color, such as grey, to one of the three colors representing the categories described above. For example, a patient whose most recent weigh-in at the doctor's office was 185 pounds, may input their weight of 185 pounds in weight input field 202. In this example, upon inputting the weight, status indicator 208 changes from a grey color to a green color, thereby alerting the patient in real time that their weight is in accordance with the baseline weight set by their healthcare provider.

In another example, the same patient may input their weight as 193 pounds. In this example, status indicator 208 will automatically change to a yellow color, indicating a weight gain that raises caution. In the example embodiment, HM computing device 1102 detects when an inputted key marker measurement may be an error by comparing the inputted measurement to the thresholds in the patient's reference model. For example, for a patient whose baseline weight is set at 180 pounds, and whose key marker measurement for the previous day was 182 pounds, may accidently enter their weight as 1,822 pounds. In this example, HM computing device 1102 compares the inputted 1,822 pounds to the thresholds in the patient's reference model and the patient's previously reported key marker measurements to determine that the inputted weight of 1,822 pounds is an error. In the example embodiment, upon detecting an error, HM computing device 1102 displays an error message on daily measurement screen 200, and prompts the patient to input their key marker measurements again.

Additionally or alternatively, HM computing device 1102 may utilize an algorithm to determine the appropriate status category for daily key marker measurements that are in-between the patient's range 210 of thresholds. In these embodiments, to determine whether a patient's key marker measurement should be categorized as baseline, warning, or critical, HM computing device 1102 may utilize an algorithm that determines the difference between the two thresholds for which the inputted measurement lies in-between, divides the difference in half, and adds the calculated value to the lower of the two thresholds to obtain a midpoint indicator. In these embodiments, HM computing device 1102 compares the inputted measurement for a specific key marker to the midpoint indicator to determine if the inputted measurement is greater than or less than the midpoint indicator. If the inputted measurement is greater than the midpoint indicator, HM computing device 1102 determines that the inputted key measurement is more than halfway towards the threshold established for the next status category. HM computing device 1102 subsequently categorizes the inputted measurement for the particular key marker in the next status category.

For example, the patient's range 210 of thresholds for weight may be 245 pounds for the baseline status category, 255 pounds for the warning status category, and 265 pounds for the critical status category. The patient may input a daily weight measurement of 251 pounds in input field 202. In this example, HM computing device 1102 may utilize the algorithm described above to determine that the inputted weight measurement of 251 pounds lies in-between the thresholds for the baseline and warning status categories. HM computing device 1102 may determine the difference of the two thresholds (245 pounds and 255 pounds), divide the difference in half, and add the calculated value to the lower of the two thresholds to obtain the midpoint indicator. In this example, the calculated value of 5 pounds is added to the threshold for the baseline status category to obtain a midpoint indicator of 250 pounds. HM computing device 1102 compares the weight measurement of 251 pounds in input field 202 to the midpoint indicator of 250 pounds to determine that the inputted weight measurement is greater than the midpoint indicator. In this example, HM computing device 1102 subsequently categorizes the inputted weight measurement in the warning status category rather than the baseline status category, and automatically changes corresponding status indicator 208 from a default color, such as grey, to a color associated with the warning status category, such as yellow.

Upon inputting measurements for each key marker, the patient selects a “next” button 212 provided on daily measurement screen 200 to provide details and answer general questions as to the patient's condition. FIG. 2B is an example screenshot of a patient question screen 250 from HM computing device 1102 for a patient user interface in accordance with an example embodiment of the present disclosure. Upon selecting “next” button 212, HM computing device 1102 displays questions 252 and 254 regarding the patient's mood/symptoms and medication. In the example embodiment, HM computing device 1102 provides a list of preset answers 256 and 258 from which the patient may choose their answers. HM computing device 1102 further provides text boxes 260 and 262 for each of the displayed questions 252 and 254 to enable the patient to provide additional details.

In the example embodiment, HM computing device 1102 displays question 252, asking how a patient is feeling at the moment the patient is inputting their daily vitals. The patient subsequently selects a response from a list of preset answers 256 provided for question 252. The patient may also choose to provide their response in textbox 260 if they would like to explain their condition. In the example embodiment, HM computing device 1102 displays question 254 with regard to any missed medications. A patient can select a response from the preset answers 258 provided for question 254, and also provide further explanations to their response in textbox 262. In further embodiments, HM computing device 1102 may display questions that are specific to each individual's disease state, medication type and dosage, and/or patient care plan. In the example embodiment, the inputted measurements and patient responses are submitted together when the patient selects “send” button 264.

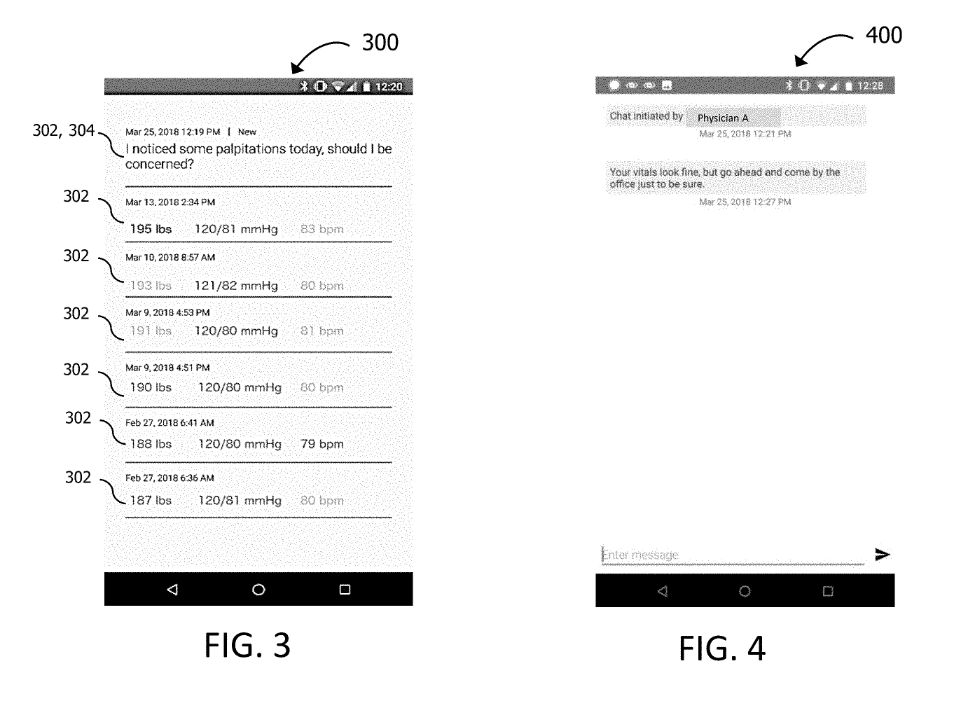

In the example embodiment, when one or more of the inputted key marker measurements is categorized as critical, HM computing device 1102 transmits an alert message (e.g., notification) to both the patient and the patient's healthcare provider. More specifically, HM computing device 1102 notifies the patient's healthcare provider of the critical status by displaying the alert message on the healthcare provider's user interface. In some embodiments, an alert message is also transmitted for key marker measurements categorized as warning. In certain embodiments, the patient's healthcare provider receives push notifications on the healthcare provider's computer device, such as healthcare provider computer device 1106 (shown in FIG. 11) when the one or more of the patient's key marker measurements are categorized as critical and/or warning. FIG. 3 is an example screenshot of a patient report screen 300 from HM computing device 1102 for a patient user interface in accordance with an example embodiment of the present disclosure. A patient can access patient report screen 300 from patient home screen 100 by selecting “my reports” button 106 (shown in FIG. 1). Patient report screen 300 displays a log of the patient's reported data. The patient's reported data includes reported entries 302 such as daily key marker measurements, questions, and/or concerns submitted by the patient via the patient's patient computer device, such as reported concern 304.

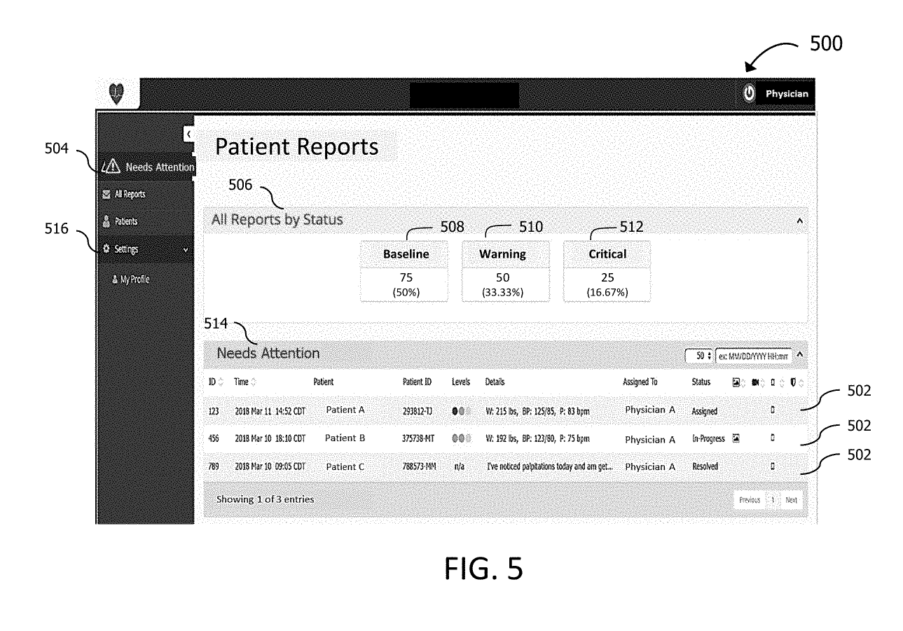

FIG. 4 is an example screenshot of a patient communication screen 400 from HM computing device 1102 for a patient user interface in accordance with an example embodiment of the present disclosure. More specifically, patient communication screen 400 (shown in FIG. 4) is a chat conversation screen initiated by the patient's healthcare provider based on a concern reported by the patient, such as reported concern 304 (shown in FIG. 3). The patient can access patient communication screen 400 from patient home screen 100 by selecting “report a concern” button 104 (shown in FIG. 1). HM computing device 1102 is configured to provide a communication channel (through patient computer device 1104 and healthcare provider computer device 1106, both shown in FIG. 11) between the patient and the patient's healthcare provider so as to enable interactive communication between the patient and the patient's healthcare provider at the patient's convenience. The communication channel may be any medium that allows the patient and the patient's healthcare provider to communicate with each other, such as via chat/text messaging, voice calls, emails, and/or video conferencing.

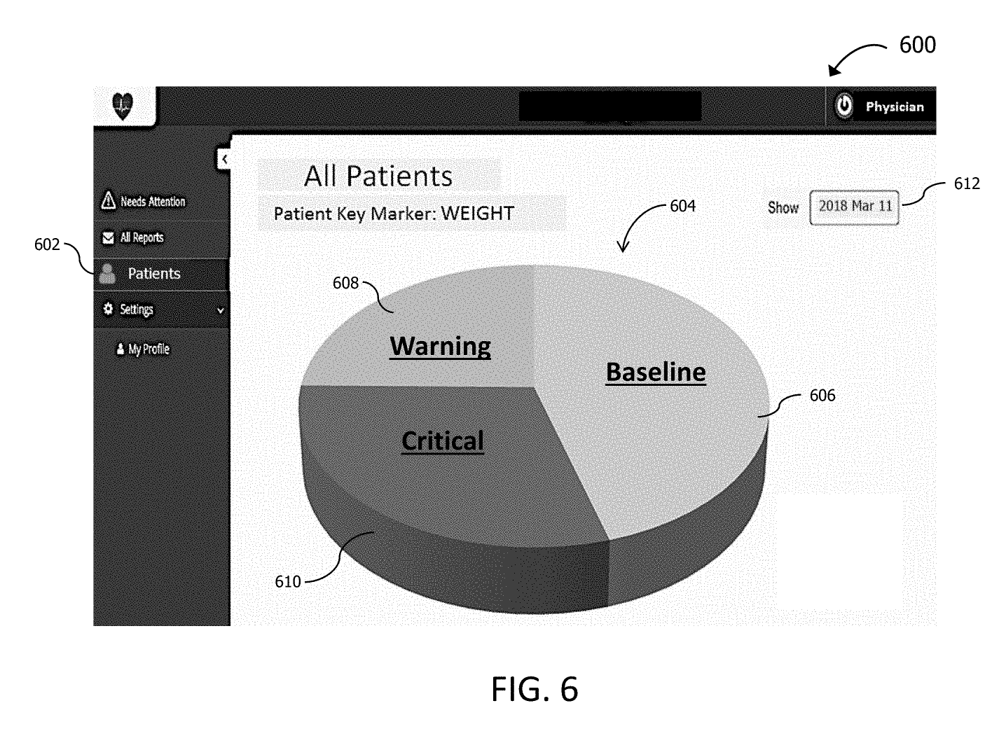

FIGS. 5-9 illustrate example screenshots of a healthcare provider user interface in accordance with example embodiments of the present disclosure. The example screenshots shown in FIGS. 5-9 may be accessed via a healthcare provider computer device, such as healthcare provider computer device 1140 (shown in FIG. 11). FIG. 5 is an example screenshot of a patient reports screen 500 from HM computing device 1102 for a healthcare provider user interface in accordance with an example embodiment of the present disclosure. More specifically, patient reports screen 500 displays patients needing attention based on reported key marker measurements. Patient reports (e.g., report entries 502) include alert messages (e.g., notifications) transmitted by HM computing device 1102 when one or more reported key marker measurement is categorized as critical. Patient reports also include questions and/or concerns submitted by a patient. In some embodiments, patient reports also include patient answers to questions provided by HM computing device 1102 in regards to reported concerns submitted by a patient. Patient reports provides healthcare providers with real time (or near real time) patient information that enables each healthcare provider to monitor their patient's condition, and act promptly when their patient is in need of immediate medical attention. Patient reports also enables healthcare providers to track each patient's reported key marker measurements and assess whether, based on a patient's recent measurements, the patient is trending toward a warning and/or critical status. In these embodiments, healthcare providers are able to take preventative measures, by reaching out to the patient and scheduling a medical visit (e.g., doctor's appointment) and/or discussing changes to the type and/or dosage of the patient's prescribed medication.

Patient reports screen 500 can be accessed by a healthcare provider by selecting a “needs attention” tab 504 provided on the healthcare provider's user interface. Patient reports screen 500 includes an “all reports by status” section 506 that includes a “baseline” tab 508, a “warning” tab 510, and a “critical” tab 512. A healthcare provider can select tabs 508, 510, and 512 to view patient reports for each status category. In some embodiments, selecting tabs 508, 510, and 512 allows a healthcare provider to view new patient reports based on patient key marker measurements for each status category. In further embodiments, selecting tabs 508, 510, and 512 causes HM computing device 1102 to display a list of patient reports for the selected status category. In other embodiments, selecting tabs 508, 510, and 512 causes HM computing device 1102 to display the patient reports in an interactive graphical form (not shown), such as a pie chart, bar chart, and/or a line graph.

Below these tabs appears a “needs attention” section 514 that includes a list of report entries 502 detailing the patients who need attention immediately. Report entries 502 include patient information such as, but not limited to, submitted date/time of the report, patient name, patient identification (“ID”) number, status indicators for each key marker such as status indicator 208 (shown in FIG. 2A), reported measurements for each key marker, the name of the healthcare provider assigned to the patient, and/or images and notes submitted by the patient in regards to their symptoms, questions, and/or concerns.

In certain embodiments, report entries 502 further includes patient answers to questions provided by HM computing device 1102 in regards to reported symptoms, questions, and/or health concerns submitted by the patient. In other embodiments, report entries 502 includes historical patient data (not shown), such as a record of the patient's past and/or upcoming (e.g., scheduled) medical visits. Medical visits may include, but are not limited to, an emergency room (“ER”) visit, a hospitalization due to a health concern and/or symptom, such as a cardiac event, and a scheduled doctor's appointment, such as a checkup or a post-hospitalization doctor's appointment. In these embodiments, a healthcare provider may quickly view all the medical visits associated with a particular patient, and select a specific medical visit, such as a prior doctor's appointment, to access data associated with that visit. A healthcare provider may access patient data, such as test results, physician comments, and prescribed prescriptions and dosages associated with a specific medical visit.

In further embodiments, report entry 502 includes an “add comment” section (not shown) that enables a healthcare provider to include additional notes and/or comments associated with a particular patient. In some embodiments, report entries 502 further includes an “initiate chat” feature (not shown) that enables a healthcare provider to reach out to a patient directly from “needs attention” section 514. For example, a healthcare provider reviewing a particular patient's report entry 502 may be concerned about the patient's reported symptoms, and subsequently select the “initiate chat” feature to engage in a secure chat session with the patient. In the example embodiment, a healthcare provider can view report entries 502 in order of time submitted or prioritized based on patients needing immediate attention. For example, the healthcare provider may choose to view report entries 502 in order of patients who have two or three key marker measurements categorized as critical as opposed to only one key marker. In this example, the healthcare provider can adjust the order of report entries 502 by selecting a “settings” tab 516 provided on the healthcare provider's user interface and/or adjusting viewing settings (not shown) provided on patient reports screen 500.

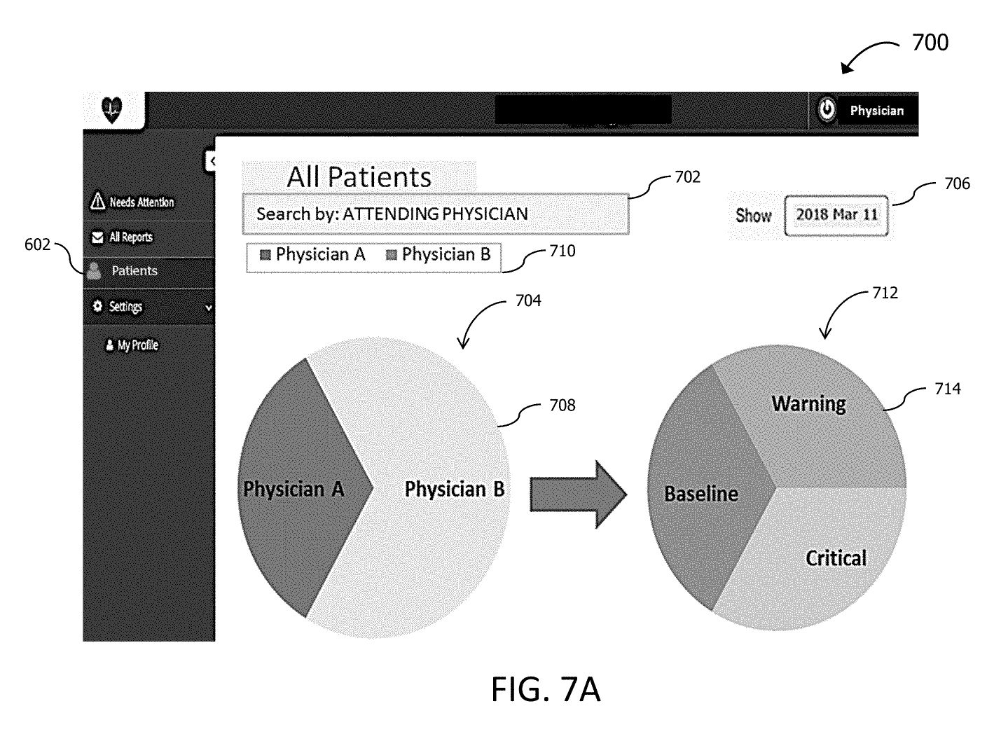

FIG. 6 is an example screenshot of a first visual representation screen 600 of patient data from HM computing device 1102 for a healthcare provider user interface in accordance with an example embodiment of the present disclosure. A healthcare provider can access first visual representation screen 600 by selecting a “patients” tab 602 provided by HM computing device 1102 in the healthcare provider user interface. First visual representation screen 600 includes an interactive graphical representation, such as a first pie chart 604. First pie chart 604 represents all patients associated with each status category (e.g., baseline, warning, and critical) for a given key marker (e.g., weight, blood pressure, and pulse). A pie chart such as first pie chart 604 is provided for each key marker. A healthcare provider who has selected “patients” tab 602 can choose to view aggregate data for all patients registered with HM computing device 1102 by key marker.

In the example embodiment, HM computing device 1102 displays first pie chart 604 for all patients based on weight. First pie chart 604 displays status category slices (e.g., portions), such as a baseline slice 606, a warning slice 608, and a critical slice 610. In the example embodiment, the status category slices are associated with a first color, a second color, and/or a third color that corresponds with each status category (e.g., green for baseline, yellow for warning, and red for critical). A healthcare provider can select one of the status category slices to view the total number of patients within the selected status category. In the example embodiment, first visual representation screen 600 includes a search function, such as search function 612, which enables a healthcare provider to filter results by time and/or date. For example, a healthcare provider may select warning slice 608 for the weight key marker to view the total number of patients needing immediate attention for a given date relative to the total number of patients registered with HM computing device 1102.

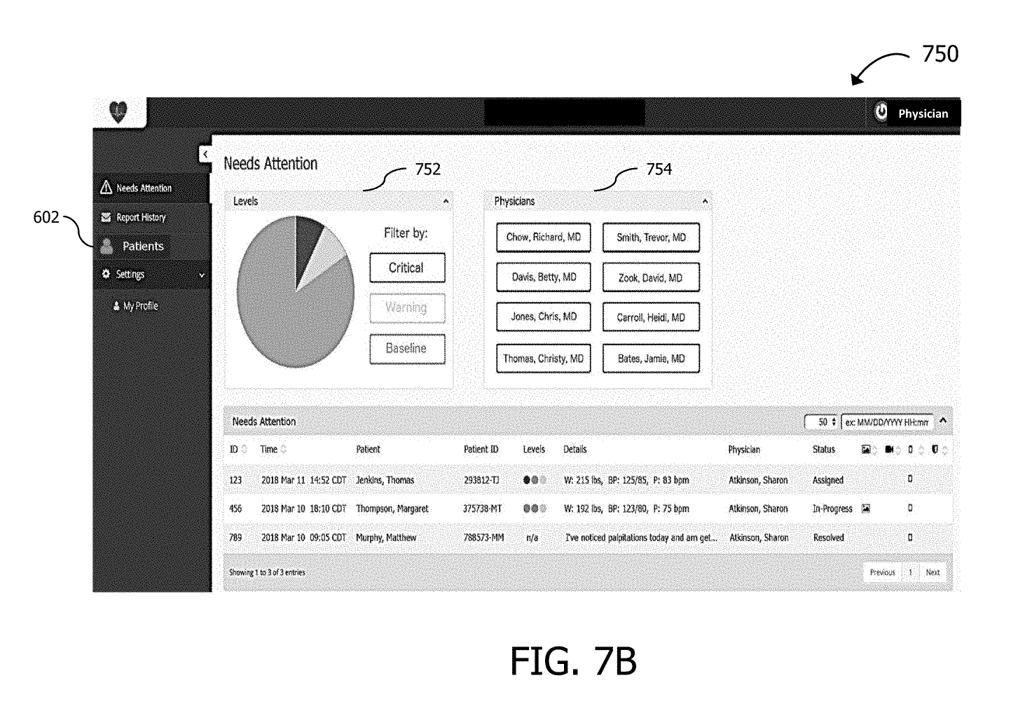

FIG. 7A is an example screenshot of a second visual representation screen 700 of patient data from HM computing device 1102 for a healthcare provider user interface in accordance with an example embodiment of the present disclosure. A healthcare provider can access second visual representation screen 700 by selecting “patients” tab 602 provided by HM computing device 1102 in the healthcare provider user interface. Second visual representation screen 700 includes a search bar 702, which enables a healthcare provider to search by, for example, attending physicians and/or nurses. Second visual representation screen 700 includes an interactive graphical representation, such as second pie chart 704 Second pie chart 704 represents all patients associated with a healthcare provider (e.g., physician, nurse, and/or healthcare professional). In the example embodiment, a healthcare provider who has selected “patients” tab 602 can choose to filter through all the patients registered with HM computing device 1102 by healthcare provider (e.g., attending physician and/or assigned nurse). Second visual representation screen 700 also includes a date filter 706 that enables a healthcare provider to view results for a specific date and/or time.

In the example embodiment, second pie chart 704 represents the total number of healthcare providers for a given medical specialty and/or department. Each slice of second pie chart 704, such as a physician slice 708, corresponds to a healthcare provider who is treating a patient registered with HM computing device 1102. For example, second pie chart 704 can display the total number of physicians in a hospital's cardiology department. In another example, second pie chart 704 can display the total number of physicians in a hospital who specialize in diagnosing and treating a specific condition, such as congestive heart failure (CHF), diabetes, chronic obstructive pulmonary disease (COPD), and various forms of cancer. In the example embodiment, each slice is associated with a color that corresponds to a specific healthcare provider. For example, a first color may be assigned to Physician A and a second color may be assigned to Physician B. In the example embodiment, a healthcare provider can view all the patients associated with a specific physician by selecting a slice that corresponds with the specific physician, such as physician slice 708.

In some embodiments, a healthcare provider can select the desired physician's name and/or assigned color in a legend 710 displayed on second visual representation screen 700. In the example embodiment, second visual representation screen 700 changes from second pie chart 704 to a third pie chart 712 when a slice from second pie chart 704, such as physician slice 708 or a physician name and/or assigned color from legend 710 is selected. Third pie chart 712 represents all the patients assigned to a given physician filtered by status category (e.g., baseline, warning, and critical). Similar to first pie chart 604 (shown in FIG. 6), third pie chart 712 includes status category slices corresponding to each status category. In some embodiments, additional information, such as the percentage of patients in each status category is displayed on each status category slice when a healthcare provider selects a status category slice. In other embodiments, selecting a slice from second pie chart 704, such as physician slice 708 causes HM computing device 1102 to display a list of patients associated with the selected physician in graphical and/or tabular form.

In further embodiments, selecting a slice on third pie chart 712 causes HM computing device 1102 to display a fourth pie chart (not shown) on second visual representation screen 700. In these embodiments, the fourth pie chart displays the number of patients in the selected status category for each key marker. Each slice on the fourth pie chart may correspond to one of weight, blood pressure, and pulse. For example, a healthcare provider who selects a warning slice 714, similar to warning slice 608 (shown in FIG. 6) on third pie chart 712, can view the key marker measurements for each patient within the warning category. In some embodiments, selecting a slice, such as physician slice 708 from second pie chart 704 causes HM computing device to display an intermediary pie chart (not shown) displaying the nurses (e.g., Registered Nurse, nurse practitioner, and/or physician's assistant) working with the selected physician. For example, the intermediary pie chart may include slices for each nurse assigned to a patient who is registered with HM computing device 1102. In these embodiments, a healthcare provider can select a slice corresponding to a specific nurse to view a pie chart similar to third pie chart 712, showing a total number of the nurse's patients in each of the three status categories.

FIG. 7B is another example screenshot 750 of second visual representation screen 700 of patient data from HM computing device 1102 for a healthcare provider user interface in accordance with an example embodiment of the present disclosure. In the example embodiment, second visual representation screen 700 includes a “levels” section 752 that displays an interactive graphical representation, such as a pie chart similar to first pie chart 604 (shown in FIG. 6). A healthcare provider can either select a color-coded slice on the pie chart or the status category name in a legend near the pie chart to view more information about the patients within each status category. Selecting a status category slice and/or name from “levels” section 752 can cause HM computing device 1102 to display aggregate patient data in the form of a second, third, and/or fourth pie chart similar to second pie chart 704 and third pie chart 712 (shown in FIG. 7). In other embodiments, selecting a status category slice and/or name may cause HM computing device 1102 to display aggregate patient data in a list and/or tabular form. In the example embodiment, second visual representation screen 700 also includes a “physicians” tab 754 that enables a healthcare provider to view all patients under a specific physician's care by selecting a displayed physician name on “physicians” tab 754.

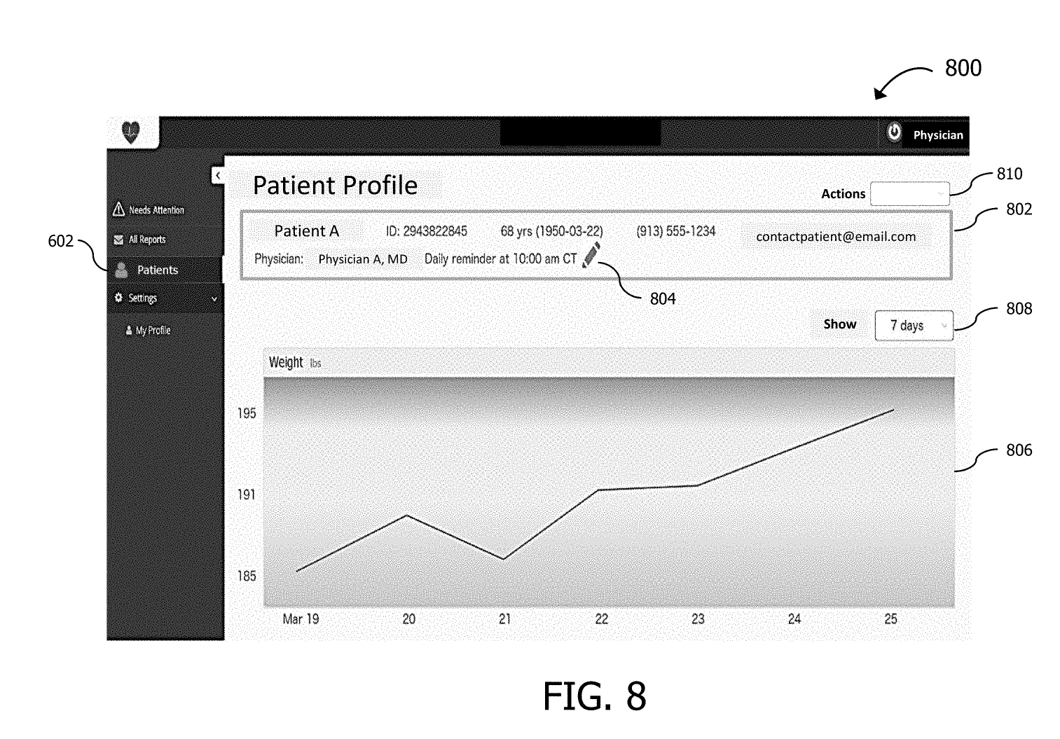

FIG. 8 is an example screenshot of a third visual representation screen 800 of an individual patient's profile from HM computing device 1102 for a healthcare provider user interface in accordance with an example embodiment of the present disclosure. A healthcare provider can access third visual representation screen 800 by selecting a patient from “patients” tab 602 provided by HM computing device 1102 in the healthcare provider user interface. More specifically, third visual representation screen 800 displays an individual patient's historical data, as opposed to the aggregate patient data displayed on first visual representation screen 600 and second visual representation screen 700 (shown in FIGS. 6 and 7).

Third visual representation screen 800 includes a patient information section 802, which includes information such as, but not limited to, patient name, patient identification (ID) number, date of birth (“DOB”), contact number, email address, attending healthcare provider information (e.g., name, contact number), and/or daily reminder(s). A healthcare provider can use an edit tool 804 provided in patient information section 802 to manage a selected patient's patient information. For example, a healthcare provider can adjust a patient's daily reminders (e.g., push notifications) from sending reminders two times a day (e.g., morning reminder and evening reminder) to four times a day by using edit tool 804. Below patient information section 802 appears a patient historical report 806 in graphical form for each key marker. In the example embodiment, patient historical report 806 displays an individual patient's reported measurements for the weight key marker within the last seven days. A healthcare provider can adjust the time period for patient historical report 806 by using a filter function 808 to view the patient's historical data for a longer or shorter period of time. HM computing device 1102 also generates and displays patient historical reports 806 for each patient's blood pressure and pulse.

Third visual representation screen 800 also includes an “actions” tab 810 that allows a healthcare provider to initiate communication with the patient and/or update the patient's charts. For example, a healthcare provider can select “actions” tab 810 to initiate a chat session with the patient, send a message and/or email to the patient, enter comments in the patient's charts, add and/or change a patient's assigned healthcare provider(s) as needed, and/or send a secure app login link to a patient via email. In certain embodiments, these actions may be available as part of “actions” tab 810 as a drop-down menu that enables a healthcare provider to select one of the actions described above. In some embodiments, instead of “actions” tab 810, third visual representation screen 800 includes a “send message” tab (not shown), an “initiate chat” tab (not shown), “comment” tab (not shown), and an “update patient data” tab (not shown). In further embodiments, a “report recap” section (not shown) appears below patient historical report 806. In these embodiments, the “report recap” section provides detailed information for each date displayed in patient historical report 806. For example, a list of reported measurements, symptoms, and/or concerns submitted by the patient may be displayed for each of the seven days shown in patient historical report 806.

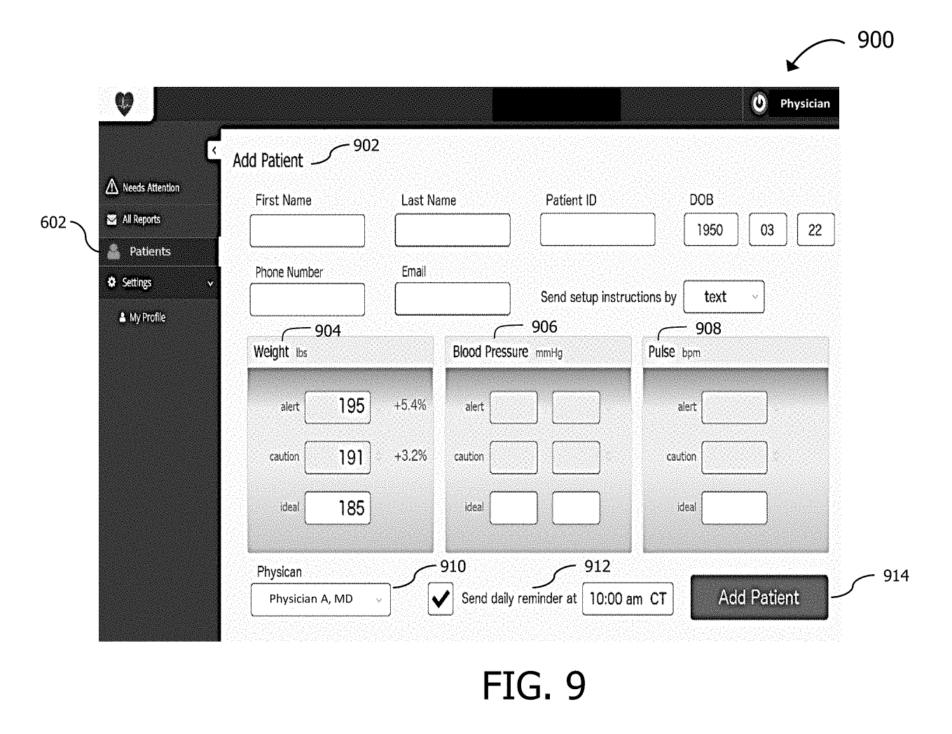

FIG. 9 is an example screenshot of a fourth visual representation screen 900 of patient data from HM computing device 1102 for a healthcare provider user interface in accordance with an example embodiment of the present disclosure. A healthcare provider can access fourth visual representation screen 900 by selecting an “add patient” tab (not shown) from “patients” tab 602 provided by HM computing device 1102 in the healthcare provider user interface. More specifically, fourth visual representation screen 900 displays patient information fields in an “add patient” section 902 for a healthcare provider to enter patient information such as, but not limited to, patient name, patient ID, DOB, and/or email address. Fourth visual representation screen 900 also includes a field that enables the healthcare provider to select how setup instructions are to be sent to the patient (e.g., by text and/or email). Below the patient information fields appear tables 904, 906, and 908 for each key marker. In the example embodiment, a healthcare provider sets thresholds for baseline (e.g., ideal), warning (e.g., caution), and critical (e.g., alert) for each key marker in their respective tables 904, 906, and 908 based on the patient's medical history. Thus, each patient registered with HM computing device 1102 has a reference model that includes the thresholds set by their healthcare provider for each key marker.

In the example embodiment, a patient's daily reported key measurements are compared to each of the established thresholds set by the patient's healthcare provider. The thresholds set for each status category can subsequently be adjusted by the healthcare provider by, for example, selecting editing tool 804 on the selected patient's patient profile, as shown by third visual representation screen 800 (shown in FIG. 8). Below tables 904, 906, and 908, HM computing device 1102 provides a “physician” tab 910 that enables a healthcare provider to select an attending physician for the patient. In other embodiments, HM computing device 1102 alternatively or additionally provides a general “healthcare provider” tab (not shown) that enables a healthcare provider to select a nurse, physician, and/or other healthcare provider for the patient.

Fourth visual representation screen 900 also includes a “daily reminder” tab 912 that enables a healthcare provider to send daily notifications (e.g., reminders) to their patient at a designated time. The daily notifications can be customized to each patient depending on their specific needs. For example, a healthcare provider may set up different types of notifications such as a medication reminder (e.g., reminding a patient to take their medication at a specific time), a measurement reminder (e.g., reminding a patient to submit their key marker measurements), and/or an appointment reminder (e.g., reminding a patient of an upcoming doctor's appointment). In the example embodiment, a healthcare provider can set up multiple reminders (e.g., morning, afternoon, evening reminders). The reminders can be in the form of push notifications displayed on the patient's patient computer device, such as patient computer device 1104 (shown in FIG. 11), emails, text messages, chats, and/or voice calls.

Fourth visual representation screen 900 further provides an “add patient” button 914 that enables a healthcare provider to register a new patient with HM computing device 1102. In further embodiments, selecting “add patient” button 914 prompts HM computing device 1102 to generate a customized registration link that enables the patient to automatically download the app associated with HM computing device 1102 onto the patient's computer device (e.g., patient computer device 1104, shown in FIG. 11). In some embodiments, HM computing device 1102 may automatically transmit the customized registration link to the patient via email. In other embodiments, HM computing device 1102 may prompt the healthcare provider registering the new patient to transmit the generated registration link to the patient via email.

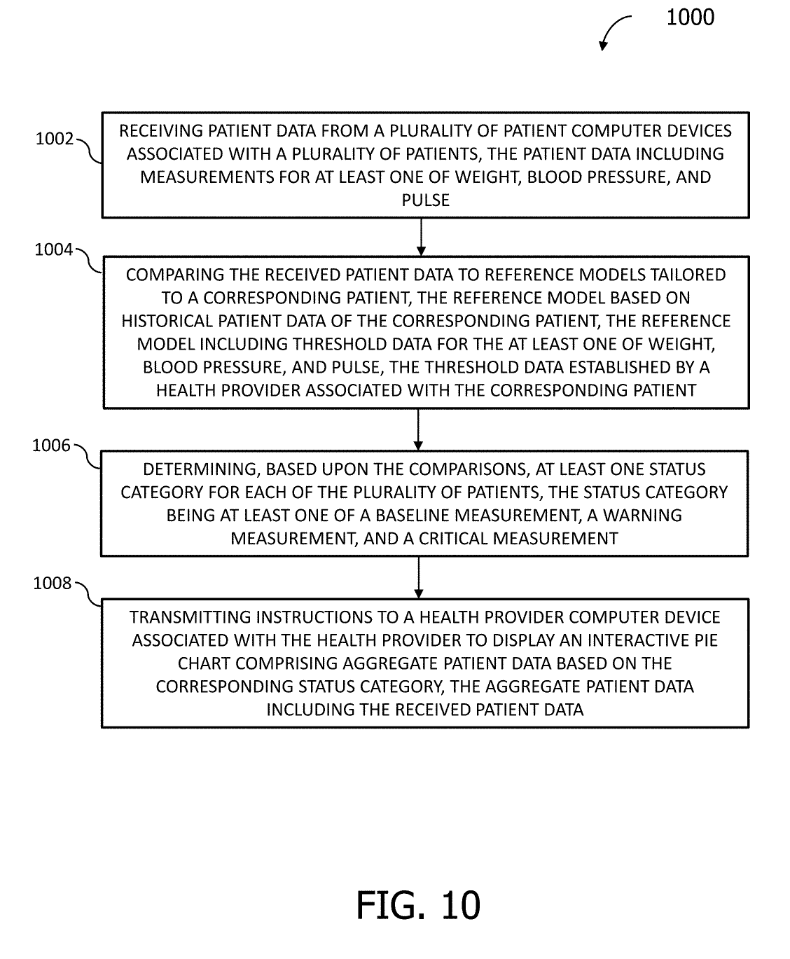

FIG. 10 is a flowchart of an example method 1000 for providing a health monitoring (HM) computer system, such as system 1100 (shown in FIG. 11). In the example embodiment, method 1000 is performed by an HM computing device, such as HM computing device 1102 (shown in FIG. 11). In certain embodiments, method 1000 may be at least partially performed by a different computing device. In other embodiments, method 1000 may include additional, fewer, or alternative actions, including those described elsewhere herein.

Method 1000 begins with the HM computing device receiving 1002 patient data from a plurality of patient computer devices associated with a plurality of patients, the patient data including measurements for at least one of weight, blood pressure, and pulse. Method 1000 also includes comparing 1004 the received patient data to reference models tailored to a corresponding patient. The reference model is based on historical patient data of the corresponding patient, and includes threshold data for the at least one of weight, blood pressure, and pulse. The threshold data is established by a healthcare provider associated with the corresponding patient. Method 1000 further includes determining 1006, based upon the comparisons, at least one status category for each of the plurality of patients. The status category is at least one of a baseline measurement, a warning measurement, and a critical measurement. Method 1000 also includes transmitting 1008 instructions to a healthcare provider computer device associated with the healthcare provider to display an interactive pie chart. The interactive pie chart comprises aggregate patient data based on the corresponding status category. The interactive pie chart further includes the received patient data.

FIG. 11 depicts a simplified block diagram of an example health monitoring (HM) system 1100 for implementing method 1000 shown in FIG. 10. In the example embodiment, system 1100 may be used to monitor a patient's vitals in real time. As described herein, health monitoring (HM) computing device 1102 may be configured to (i) receive patient data from a plurality of patient computer devices associated with a plurality of patients, the patient data including measurements for at least one of weight, blood pressure, and pulse; (ii) compare the received patient data to reference models tailored to a corresponding patient, the reference model based on historical patient data of the corresponding patient, the reference model including threshold data for the at least one of weight, blood pressure, and pulse, the threshold data established by a healthcare provider associate with the corresponding patient; (iii) determine, based upon the comparisons, at least one status category for each of the plurality of patients, the status category being at least one of a baseline measurement, a warning measurement, and a critical measurement; and/or (iv) transmit instructions to a healthcare provider computer device associated with the healthcare provider to display an interactive pie chart comprising aggregate patient data based on the corresponding status category, the aggregate patient data including the received patient data, as described herein.

In the example embodiment, patient computer devices 1104 and healthcare provider computer devices 1106 are computers that include a web browser or a software application, which enables patient computer devices 1104 to access remote computer devices, such as HM computing device 1102 using the Internet or other network. More specifically, patient computer devices 1104 and healthcare provider computer devices 1106 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. Patient computer devices 1104 and healthcare provider computer devices 1106 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

A database server 1108 is communicatively coupled to a database 1110 that stores data. In one embodiment, database 1110 may include reported key marker measurements for each patient registered with HM computing device 1102, reference models for each corresponding patient, patient charts and/or medical records (e.g., historical patient data), patient information, and aggregate patient data. In some embodiments, database 1110 is stored remotely from HM computing device 1102. In some embodiments, database 1110 is decentralized. In some embodiments, database 1110 is a cloud or server-based electronic medical record (“EMR”) system. In the example embodiment, a patient, may access database 1110 via patient computer device 1104 by logging onto HM computing device 1102, as described herein. A healthcare provider may also access database 1110 via healthcare provider computer device 1106 by logging onto HM computing device 1102, as described herein. In some embodiments, database 1110 includes any computer server, cloud or other digital data storage device.

HM computing device 1102 may be in communication with a plurality of patient computer devices 1104, a plurality of healthcare provider computer devices 1106, and a plurality of insurer network 1112 computer devices to monitor and transmit data associated with patient vitals in real time. In some embodiments, HM computing device 1102 tracks the amount of time each patient uses the app on their respective patient computer device 1104 to submit daily key marker measurements, submit patient information and/or concerns, answer questions provided by HM computing device 1102 with regards to submitted concerns, and engage in a chat session with a healthcare provider. Additionally or alternatively, HM computing device 1102 automatically tracks the amount of time each healthcare provider spends reviewing and evaluating patient data on their respective healthcare provider computing device 1140. For example, HM computing device 1102 may track the amount of time a healthcare provider spent reviewing data associated with a specific patient and/or a group of patients. In another example, HM computing device 1102 may track the amount of time spent by a healthcare provider in communicating with a patient (e.g., engaging in a chat session).

In further embodiments, HM computing device 1102 transmits, to computer devices associated with health insurance companies (not shown), data associated with the amount of time spent by each healthcare provider in evaluating patient(s). For example, the amount of time spent by a healthcare provider in evaluating a particular patient may be reported by HM computing device 1102 to the patient's health insurance company for billing and payment purposes with regard to the provided telehealth (e.g., telemedicine) services.

In the example embodiment, HM computing device 1102 is a remote computing device accessed in the execution of an application installed on patient computer device 1104. For example, HM computing device 1102 may be associated with a hospital and/or a health system, and be accessed during the execution of a hospital application. In the example embodiment, HM computing device 1102 is a computer that allows remote computers that include a web browser or a software application, such as patient computer devices 1104 and healthcare provider computer devices 1106, to access for communication, using the Internet or other network. More specifically, HM computing device 1102 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. HM computing device 1102 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

In some embodiments, HM computing device 1102 may transmit patient health data, such as, but not limited to, reported key marker measurements, determined status categories for a given patient's reported measurements, aggregate patient data displayed in graphical and/or tabular form, and/or other patient-related information to a remote computing device such as insurer network 1112 computer devices and healthcare provider computer devices 1140.

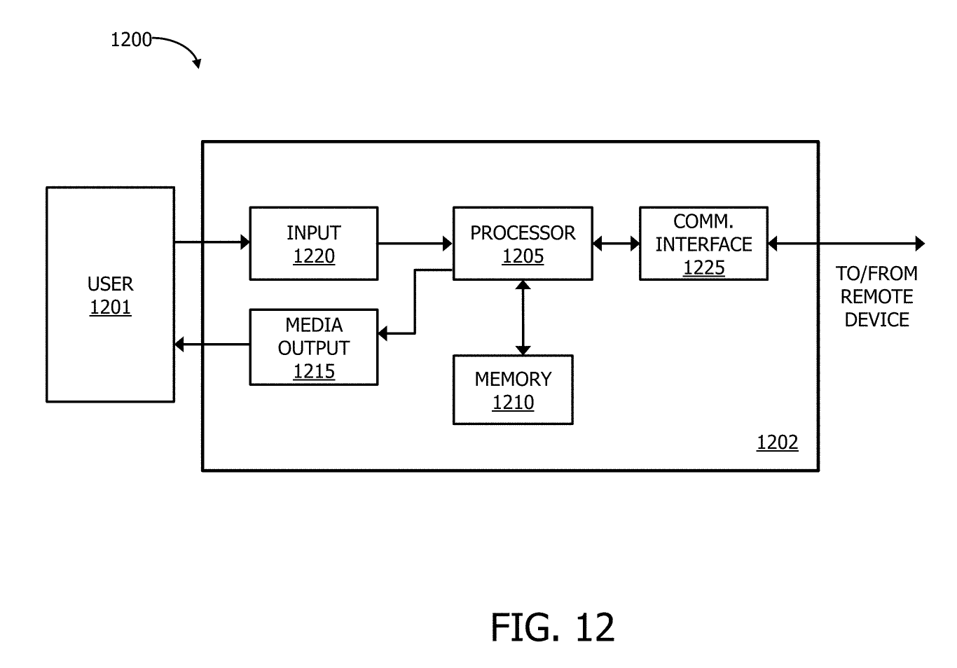

FIG. 12 depicts an example configuration of a remote or user computer device 1202, such as patient computer device 1104, healthcare provider computer device 1140, insurer network 1112 computer device (all shown in FIG. 11), in accordance with one embodiment of the present disclosure. User computer device 1202 may be operated by a user 1201. User computer device 1202 may include a processor 1205 for executing instructions. In some embodiments, executable instructions may be stored in a memory area 1210. Processor 1205 may include one or more processing units (e.g., in a multi-core configuration). Memory area 1210 may be any device allowing information such as executable instructions and/or transaction data to be stored and retrieved. Memory area 1210 may include one or more computer readable media.

User computer device 1202 may also include at least one media output component 1215 for presenting information to user 1201. Media output component 1215 may be any component capable of conveying information to user 1201. In some embodiments, media output component 1215 may include an output adapter (not shown) such as a video adapter and/or an audio adapter. An output adapter may be operatively coupled to processor 1205 and operatively coupleable to an output device such as a display device (e.g., a cathode ray tube (CRT), liquid crystal display (LCD), light emitting diode (LED) display, or “electronic ink” display) or an audio output device (e.g., a speaker or headphones).

In some embodiments, media output component 1215 may be configured to present a graphical user interface (e.g., a web browser and/or a client application) to user 1201. A graphical user interface may include, for example, a patient user interface and/or a healthcare provider user interface for accessing a health monitoring application. In some embodiments, user computer device 1202 may include an input device 1220 for receiving input from user 1201. User 1201 may use input device 1220 to, without limitation, provide and/or access a patient's daily key marker measurements, questions, and/or concerns.

Input device 1220 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 1215 and input device 1220.

User computer device 1202 may also include a communication interface 1225, communicatively coupled to a remote device such as HM computing device 1102 (shown in FIG. 11). Communication interface 1225 may include, for example, a wired or wireless network adapter and/or a wireless data transceiver for use with a mobile telecommunications network.

Stored in memory area 1210 are, for example, computer readable instructions for providing a user interface to user 1201 via media output component 1215 and, optionally, receiving and processing input from input device 1220. A user interface may include, among other possibilities, a web browser and/or a client application. Web browsers enable users, such as user 1201, to display and interact with media and other information typically embedded on a web page or a website from HM computing device 1102. A client application may allow user 1201 to interact with, for example, HM computing device 1102. For example, instructions may be stored by a cloud service, and the output of the execution of the instructions sent to the media output component 1215.

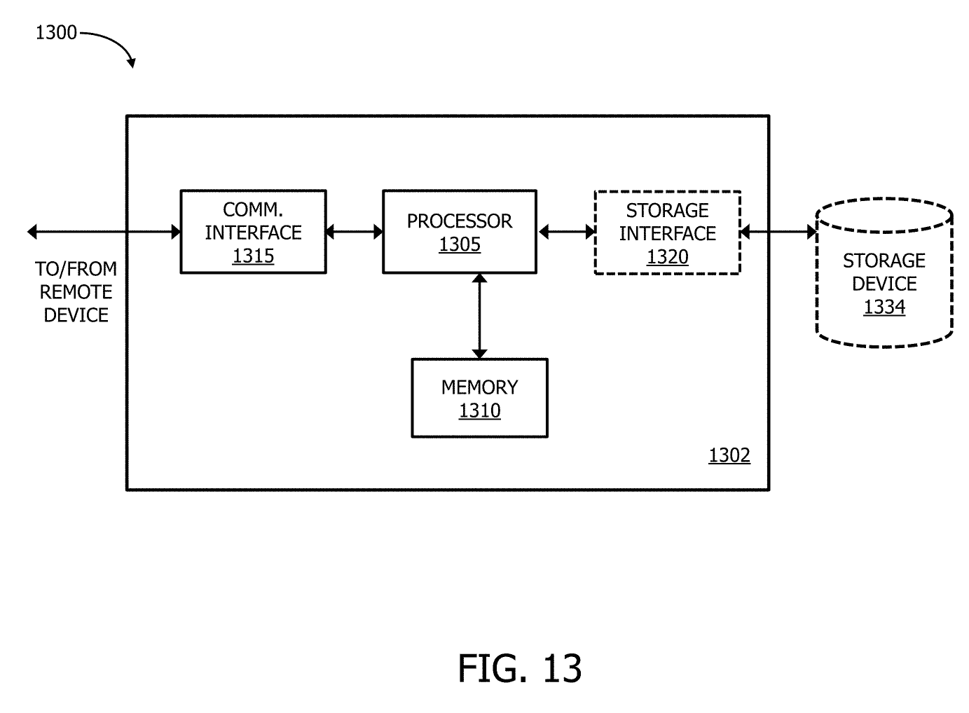

FIG. 13 depicts an example configuration of a server computing device 1302, such as HM computing device 1102, insurer network 1112, and database server 1108 (all shown in FIG. 11), in accordance with one embodiment of the present disclosure. Server computer device 1302 may also include a processor 1305 for executing instructions. Instructions may be stored in a memory area 1310. Processor 1305 may include one or more processing units (e.g., in a multi-core configuration).

Processor 1305 may be operatively coupled to a communication interface 1315 such that server computer device 1302 is capable of communicating with a remote device such as another server computer device 1302, HM computing device 1102, patient computer devices 1104, healthcare provider computer devices 1140, and insurer network 1112 computer devices (all shown in FIG. 11) (for example, using wireless communication or data transmission over one or more radio links or digital communication channels). For example, communication interface 1315 may receive questions, concerns, and/or daily key marker measurements from patient computer devices 1104 via the Internet, as illustrated in FIGS. 2-4.

Processor 1305 may also be operatively coupled to a storage device 1334. Storage device 1334 may be any computer-operated hardware suitable for storing and/or retrieving data, such as, but not limited to, data associated with database 1110 (shown in FIG. 11). In some embodiments, storage device 1334 may be integrated in server computer device 1302. For example, server computer device 1302 may include one or more hard disk drives as storage device 1334.

In other embodiments, storage device 1334 may be external to server computer device 1302 and may be accessed by a plurality of server computer devices 1302. For example, storage device 1334 may include a storage area network (SAN), a network attached storage (NAS) system, and/or multiple storage units such as hard disks and/or solid state disks in a redundant array of inexpensive disks (RAID) configuration.

In some embodiments, processor 1305 may be operatively coupled to storage device 1334 via a storage interface 1320. Storage interface 1320 may be any component capable of providing processor 1305 with access to storage device 1334. Storage interface 1320 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 1305 with access to storage device 1334.

Processor 1305 may execute computer-executable instructions for implementing aspects of the disclosure. In some embodiments, the processor 1305 may be transformed into a special purpose microprocessor by executing computer-executable instructions or by otherwise being programmed. For example, the processor 1305 may be programmed with the instructions.

At least one of the technical solutions to the technical problems provided by this system may include: (i) accurately monitoring patients in real-time by determining status categories (e.g., baseline, warning, and critical) based on vital measurements (e.g., key marker measurements) provided by a patient; (ii) facilitating communication between a patient and their healthcare provider; (iii) providing interactive visual representations (e.g., pie chart) of aggregate patient data to allow a healthcare provider to easily filter all patients by status categories associated with each key marker; (iv) providing a patient alert system that notifies both a patient and their healthcare provider that the patient needs to seek immediate medical attention; and/or (v) improving continuous patient data collection for use by a healthcare provider and/or an insurer provider.

The methods and systems described herein may be implemented using computer programming or engineering techniques including computer software, firmware, hardware, or any combination or subset thereof, wherein the technical effects may be achieved by performing at least one of the following steps: (i) receive patient data from a plurality of patient computer devices associated with a plurality of patients, the patient data including measurements for at least one of weight, blood pressure, and pulse; (ii) compare the received patient data to reference models tailored to a corresponding patient, the reference model based on historical patient data of the corresponding patient, the reference model including threshold data for the at least one of weight, blood pressure, and pulse, the threshold data established by a healthcare provider associated with the corresponding patient; (iii) determine, based upon the comparisons, at least one status category for each of the plurality of patients, the status category being at least one of a baseline measurement, a warning measurement, and a critical measurement; and/or (iv) transmit instructions to a healthcare provider computer device associated with the healthcare provider to display an interactive pie chart comprising aggregate patient data based on the corresponding status category, the aggregate patient data including the received patient data.

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object-oriented databases, and any other structured or unstructured collection of records or data that is stored in a computer system. The above examples are not intended to limit in any way the definition and/or meaning of the term database. Examples of RDBMS's include, but are not limited to, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, Calif.; IBM is a registered trademark of International Business Machines Corporation, Armonk, N.Y.; Microsoft is a registered trademark of Microsoft Corporation, Redmond, Wash.; and Sybase is a registered trademark of Sybase, Dublin, Calif.)

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In another embodiment, a computer program is provided, and the program is embodied on a computer-readable medium. In an example embodiment, the system is executed on a single computer system, without requiring a connection to a server computer. In a further example embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, Calif.). In yet a further embodiment, the system is run on a Mac OS® environment (Mac OS is a registered trademark of Apple Inc. located in Cupertino, Calif.). In still yet a further embodiment, the system is run on Android® OS (Android is a registered trademark of Google, Inc. of Mountain View, Calif.). In another embodiment, the system is run on Linux® OS (Linux is a registered trademark of Linus Torvalds of Boston, Mass.). The application is flexible and designed to run in various different environments without compromising any major functionality.

In some embodiments, the system includes multiple components distributed among a plurality of computer devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes. The present embodiments may enhance the functionality and functioning of computers and/or computer systems.

As used herein, an element or step recited in the singular and preceded by the word “a” or “an” should be understood as not excluding plural elements or steps, unless such exclusion is explicitly recited. Furthermore, references to “example embodiment,” “example embodiment,” or “one embodiment” of the present disclosure are not intended to be interpreted as excluding the existence of additional embodiments that also incorporate the recited features.

Furthermore, as used herein, the term “real-time” refers to at least one of the time of occurrence of the associated events, the time of measurement and collection of predetermined data, the time to process the data, and the time of a system response to the events and the environment. In the embodiments described herein, these activities and events occur substantially instantaneously.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A health monitoring (HM) computing device for monitoring patient vitals in real time, the HM computing device including at least one processor in communication with at least one memory device, the at least one processor programmed to:
receive patient data from a plurality of user computer devices associated with a plurality of patients, the patient data including measurements for at least one of weight, blood pressure, and pulse;
for each patient of the plurality of patients, compare the received patient data corresponding to the patient to a reference model tailored to the corresponding patient, the reference model based on historical patient data of the corresponding patient, and including threshold data for the at least one of weight, blood pressure, and pulse, the threshold data established by a healthcare provider associated with the corresponding patient;
determine, based upon the comparisons, at least one status category for each of the plurality of patients;
generate instructions for displaying a first graphical user interface on a healthcare provider computer device associated with the healthcare provider; and
transmit the generated instructions to the healthcare provider computer device to cause the first graphical user interface to be displayed on the healthcare provider computer device.

2. The HM computing device of claim 1, wherein the at least one status category is at least one of a baseline measurement status category, a warning measurement status category, and a critical measurement status category.

3. The HM computing device of claim 1, wherein the first graphical user interface includes an interactive pie chart comprising aggregate patient data based on the corresponding status category, the aggregate patient data including the received patient data.

4. The HM computing device of claim 1, wherein a plurality of status categories are determined for each patient, wherein each status category corresponds to measurements received for each of weight, blood pressure, and pulse.

5. The HM computing device of claim 1, wherein the reference model includes a plurality of thresholds based on the threshold data established for each of the plurality of patients, the plurality of thresholds including:
a first threshold corresponding to a baseline measurement status category,
a second threshold corresponding to a warning measurement status category, and
a third threshold corresponding to a critical measurement status category.

6. The HM computing device of claim 5, wherein the at least one processor is further programmed to determine at least one status category for patient data that lies in-between two of the plurality of thresholds by:
determining a calculated value;
calculating a midpoint indicator by adding the determined value to a lower threshold of the two thresholds; and
comparing the received patient data to the midpoint indicator to determine the at least one status category for the received patient data, wherein the at least one status category corresponds to one of the two thresholds.

7. The HM computing device of claim 6, wherein the calculated value is determined by:
calculating a difference between the two thresholds for which the received patient data is in-between, wherein one of the two thresholds has a lower threshold value than the other threshold; and
dividing the calculated difference by two.

8. The HM computing device of claim 1, wherein the first graphical user interface includes an interactive pie chart having a plurality of slice portions, the plurality of slice portions including:
a first slice portion corresponding to a baseline measurement status category, the first slice portion representing patients having measurements within the baseline measurement status category;
a second slice portion corresponding to a warning measurement status category, the second slice portion representing patients having measurements within the warning measurement status category; and
a third slice portion corresponding to a critical measurement status category, the third slice portion representing patients having measurements within the critical measurement status category.

9. The HM computing device of claim 8, wherein
the first slice portion is associated with a first color corresponding to the baseline measurement status category;
the second slice portion is associated with a second color corresponding to the warning measurement status category; and
the third slice portion is associated with a third color corresponding to the critical measurement status category,
wherein the first color, the second color, and the third color are each different from one another.

10. The HM computing device of claim 8, wherein, upon receiving a user selection of one of the plurality of slice portions, the at least one processor is further programmed to:
generate instructions for displaying a second graphical user interface on the healthcare provider computer device, the second graphical user interface including a total number of patients within the corresponding status category and patient data for each patient within the corresponding status category; and
transmit the generated instructions to the healthcare provider computer device to cause the second graphical user interface to be displayed on the healthcare provider computer device.

11. The HM computing device of claim 1, wherein the at least one processor is further programmed to:
generate instructions for displaying a second graphical user interface on a patient computer device associated with each of the plurality of patients, the second graphical user interface including:
a first input section for inputting patient measurements corresponding to weight;
a second input section for inputting patient measurements corresponding to blood pressure; and
a third input section for inputting patient measurements corresponding to pulse,
wherein each input section includes a status indicator configured to alert each of the plurality of patients as to whether the inputted patient measurements for a corresponding input section is within at least one of a baseline measurement status category, a warning measurement status category, and a critical measurement status category; and
transmit the generated instructions to each patient computer device to cause the second graphical user interface to be displayed on each corresponding patient computer device.

12. The HM computing device of claim 11, wherein the status indicator of each input section is instructed to change from a first color to a second color to alert each of the plurality of patients in real time.

13. The HM computing device of claim 11, wherein the at least one processor is further programmed to:
generate instructions for displaying a third graphical user interface on the patient computer device associated with each of the plurality of patients, the third graphical user interface including a general question section that includes at least one question prompt, a corresponding list of answer options below the question prompt, and a text box positioned below the corresponding list of answer options.

14. A computer-based method for monitoring patient vitals in real time, the method implemented using a health monitoring (HM) computing device, wherein the HM computing device comprises at least one processor in communication with at least one memory device, the method comprising:
receiving, by the HM computing device, patient data from a plurality of user computer devices associated with a plurality of patients, the patient data including measurements for at least one of weight, blood pressure, and pulse;
for each patient of the plurality of patients, comparing, by the HM computing device, the received patient data corresponding to the patient to a reference model tailored to the corresponding patient, the reference model based on historical patient data of the corresponding patient, and including threshold data for the at least one of weight, blood pressure, and pulse, the threshold data established by a healthcare provider associated with the corresponding patient;
determining, based upon the comparisons, at least one status category for each of the plurality of patients;
generating instructions for displaying a first graphical user interface on a healthcare provider computer device associated with the healthcare provider; and
transmitting the generated instructions to the healthcare provider computer device to cause the first graphical user interface to be displayed on the healthcare provider computer device.

15. The computer-implemented method of claim 14, wherein the first graphical user interface includes an interactive pie chart having a plurality of slice portions, the plurality of slice portions including:
a first slice portion corresponding to a baseline measurement status category, the first slice portion representing patients having measurements within the baseline measurement status category;
a second slice portion corresponding to a warning measurement status category, the second slice portion representing patients having measurements within the warning measurement status category; and
a third slice portion corresponding to a critical measurement status category, the third slice portion representing patients having measurements within the critical measurement status category.

16. One or more non-transitory computer-readable storage media having computer-executable instructions embodied thereon, wherein, when executed by at least one processor on a health monitoring (HM) computing device, the computer-executable instructions cause the at least one processor to:
receive patient data from a plurality of user computer devices associated with a plurality of patients, the patient data including measurements for at least one of weight, blood pressure, and pulse;
for each patient of the plurality of patients, compare the received patient data corresponding to the patient to a reference model tailored to the corresponding patient, the reference model based on historical patient data of the corresponding patient, and including threshold data for the at least one of weight, blood pressure, and pulse, the threshold data established by a healthcare provider associated with the corresponding patient;
determine, based upon the comparisons, at least one status category for each of the plurality of patients;
generate instructions for displaying a first graphical user interface on a healthcare provider computer device associated with the healthcare provider; and
transmit the generated instructions to the healthcare provider computer device to cause the first graphical user interface to be displayed on the healthcare provider computer device.

17. The one or more non-transitory computer-readable storage media of claim 16, wherein the reference model includes a plurality of thresholds based on the threshold data established for each of the plurality of patients, the plurality of thresholds including:
a first threshold corresponding to a baseline measurement status category,
a second threshold corresponding to a warning measurement status category, and
a third threshold corresponding to a critical measurement status category.

18. The one or more non-transitory computer-readable storage media of claim 17 having computer-executable instructions embodied thereon, wherein, when executed by the at least one processor on the HM computing device, the computer-executable instructions further cause the at least one processor to determine at least one status category for patient data that lies in-between two of the plurality of thresholds by:
determining a calculated value;
calculating a midpoint indicator by adding the determined value to a lower threshold of the two thresholds; and
comparing the received patient data to the midpoint indicator to determine the at least one status category for the received patient data, wherein the at least one status category corresponds to one of the two thresholds.

19. The one or more non-transitory computer-readable storage media of claim 16, wherein the first graphical user interface includes an interactive pie chart having a plurality of slice portions, the plurality of slice portions including:
a first slice portion corresponding to a baseline measurement status category, the first slice portion representing patients having measurements within the baseline measurement status category;
a second slice portion corresponding to a warning measurement status category, the second slice portion representing patients having measurements within the warning measurement status category; and
a third slice portion corresponding to a critical measurement status category, the third slice portion representing patients having measurements within the critical measurement status category.

20. The one or more non-transitory computer-readable storage media of claim 16 having computer-executable instructions embodied thereon, wherein, when executed by the at least one processor on the HM computing device, the computer-executable instructions further cause the at least one processor to:
generate instructions for displaying a second graphical user interface on a patient computer device associated with each of the plurality of patients, the second graphical user interface including:
a first input section for inputting patient measurements corresponding to weight;
a second input section for inputting patient measurements corresponding to blood pressure; and
a third input section for inputting patient measurements corresponding to pulse,
wherein each input section includes a status indicator configured to alert each of the plurality of patients as to whether the inputted patient measurements for a corresponding input section is within at least one of a baseline measurement status category, a warning measurement status category, and a critical measurement status category; and
transmit the generated instructions to each patient computer device to cause the second graphical user interface to be displayed on each corresponding patient computer device.
